(19) United States
(12) Patent Application Publication
(10) Pub. No.: US 2012/0150019 A1
(43) Pub. Date: Jun. 14, 2012

(54) MRI BY DIRECT TRANSVERSE HYPERPOLARIZATION USING LIGHT ENDOWED WITH ORBITAL ANGULAR MOMENTUM

(75) Inventors: Daniel Elgort, New York, NY (US); Remus Albu, Forest Hills, NY (US)

(73) Assignee: KONINKLIJKE PHILIPS ELECTRONICS N.V., EINDHOVEN (NL)

(21) Appl. No.: 13/386,676

(22) PCT Filed: Jul. 9, 2010
(86) PCT No.: PCT/IB10/53150

§ 371 Date: Jan. 24, 2012

Related U.S. Application Data
(60) Provisional application No. 61/232,817, filed on Aug. 11, 2009.
(60) Provisional application No. 61/348,761, filed on May 27, 2010.

(51) Int. Cl. A61B 5/055

(52) U.S. Cl. 600/411; 600/410; 600/421

(57) ABSTRACT

A magnetic resonance system includes a main magnet (12,12′, 12″) which generates a static magnetic field B₀ in an examination region (14,14′,14″). A hyperpolarization device (26,26′,26″) directly hyperpolarizes nuclear spins via electromagnetic radiation endowed with orbital angular momentum transverse to the static magnetic field B₀ for inducing magnetic resonance. The hyperpolarization device includes an orientation tracking unit (100) which determines an orientation of the endowed photon beam relative to a predefined external coordinate system. An orientation modifier (104) adjusts the orientation of the endowed photon beam to an optimal orientation according to the determined relative orientation.

The present application relates to the magnetic resonance arts. It finds particular application in magnetic resonance imaging (MRI) and spectroscopy (MRS), and will be described with particular reference thereto.

Conventional magnetic resonance imaging (MRI) and spectroscopy (MRS) systems use powerful static magnetic field, generally referred to as B₀, to polarize the spin vector of protons, particularly protons inside the nuclei of water and other molecules, thereby creating a signal that is appropriate for imaging and chemical analysis. Using RF excitation pulses, the system knocks the spin vectors out of alignment, and as they precess into realignment, that is, resonate, they produce a resonance signal that is used for imaging. This approach, however, only enables MRI scanners to achieve a net polarization from a small fraction of the water protons; for example, a 1.5 Tesla magnetic field, at room temperature, will polarize approximately 0.0005% of the protons. The MR system then uses a transverse magnetic field, generally referred to as B₁, oscillating in the radiofrequency (RF) band to excite the polarized nuclei by rotating them out of alignment of the B₀ field. Once the B₁ field is removed, the excited polarized nuclei relax into alignment with the B₀ and while doing so emit a MR signal. The resonating dipoles are exposed to gradient magnetic fields to localize the resultant resonance relaxation signals. The resonance relaxation signals are received and reconstructed into a single or multiple dimension image, for example.

Magnetic resonance (MR) systems characteristically include an RF transmitter, generally an RF generator coupled to a transmit coil, that generates a B₁ magnetic field tuned to the Larmor frequency of the nuclear species of interest that excites the polarized nuclear species. A drawback of conventional systems is that state of the art RF transmitters are not able to achieve a uniform excitation in the imaging volume. A B₁ magnetic field that is designed to achieve a given excitation angle, actually imparts a distribution of excitation angles. This limitation of B₁ field excitations reduces the maximum achievable signal-to-noise (SNR) and leads to the generation of stimulated echoes that can cause severe image artifacts.

Additionally, B₁ magnetic fields are unable to effectively excite multiple molecular and nuclear species simultaneously. In most imaging sequences, the B₁ magnetic field is designed to excite the hydrogen protons in water molecules, which requires the B₁ field to be tuned to a specific frequency, i.e. 64 MHz for a 1.5 Tesla system. However, simultaneous excitation of multiple atomic species, e.g. Carbon (¹³C), Oxygen (¹⁷O), Nitrogen (¹⁴N), and Phosphorus (³¹P), is not possible using the B₁ magnetic fields generated by most commercial MR systems. Furthermore, spatial encoding of multiple molecular and atomic species is not possible in conventional systems. If multiple forms of a molecular species with distinct chemical shifts, e.g. ¹H in water and ¹H in lipids, that exists outside the intended imaging plane, they will be mistakenly excited.

Another issue is that the B₁ excitation field is many times stronger than the resonance signal, but with a common frequency spectrum. Complex systems are employed to protect the resonance signal receiving circuitry from the B₁ excitation field.

The present application provides a new and improved magnetic resonance system which overcomes the above-referenced problems and others.

In accordance with one aspect, a magnetic resonance system includes a main magnet which generates a static magnetic field B₀ in an examination region. A hyperpolarization device directly hyperpolarizes nuclear spins via electromagnetic radiation endowed with orbital angular momentum. The nuclear spins are hyperpolarized transverse to the static magnetic field B₀ for inducing magnetic resonance.

In accordance with another aspect, a method for magnetic resonance includes generating a static magnetic field B₀ through an examination region to polarize dipoles and inducing resonance in the polarized dipoles via electromagnetic radiation endowed with orbital angular momentum.

One advantage resides in higher signal-to-noise ratio.

Another advantage resides in increased sampling rate.

Another advantage resides in improved patient safety.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

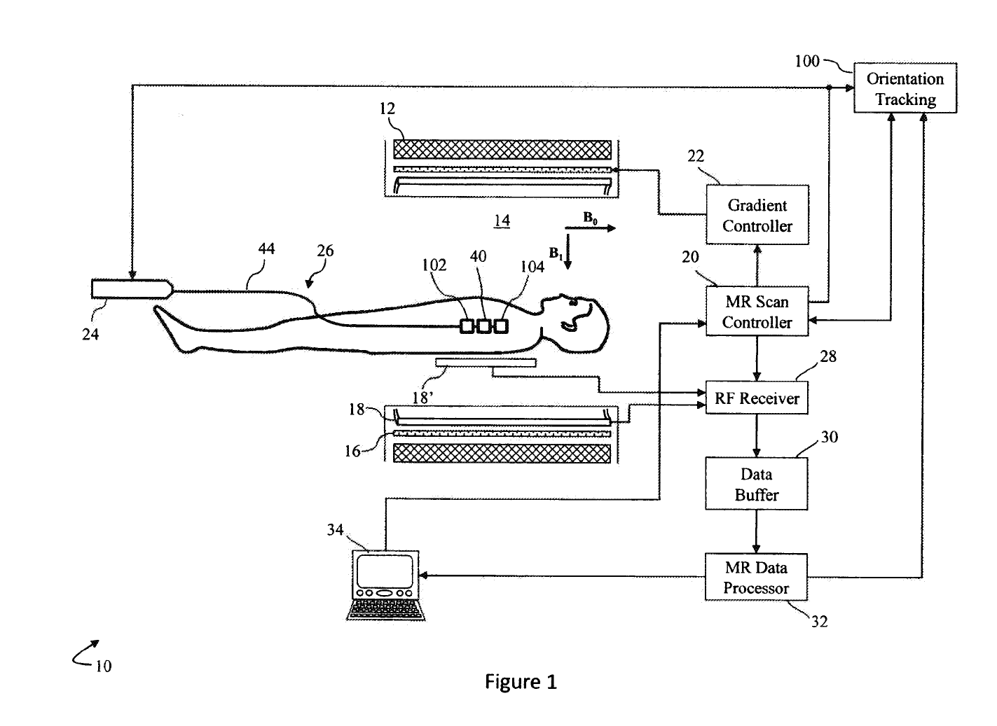

FIG. 1 is a diagrammatic illustration of magnetic resonance system including a hyperpolarization device;

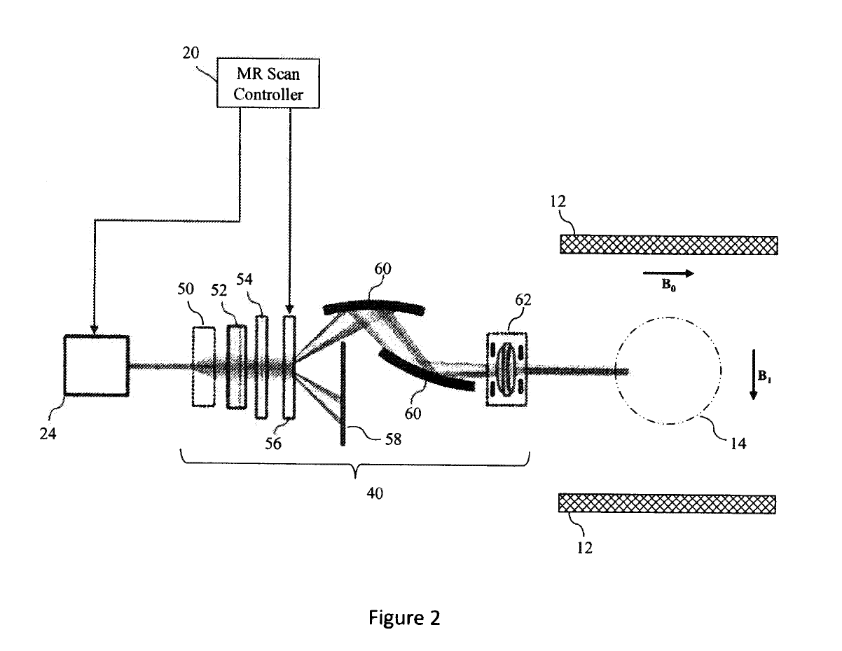

FIG. 2 is a diagrammatic illustration of an embodiment of an endowment arrangement of the hyperpolarization device;

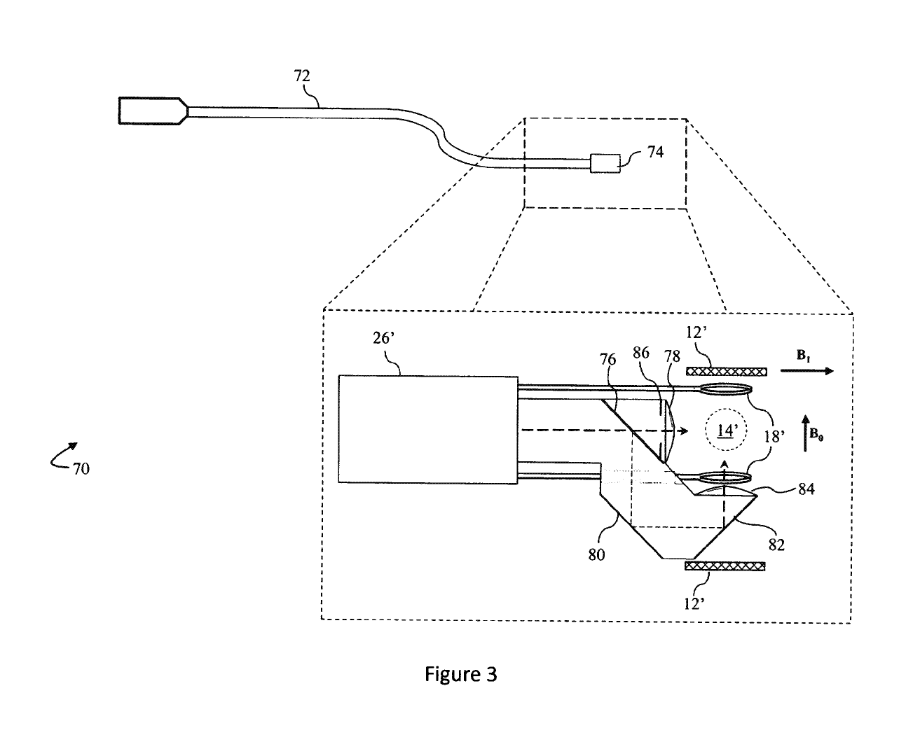

FIG. 3 is a diagrammatic illustration of one embodiment of a hyperpolarization device entirely contained within an invasive device;

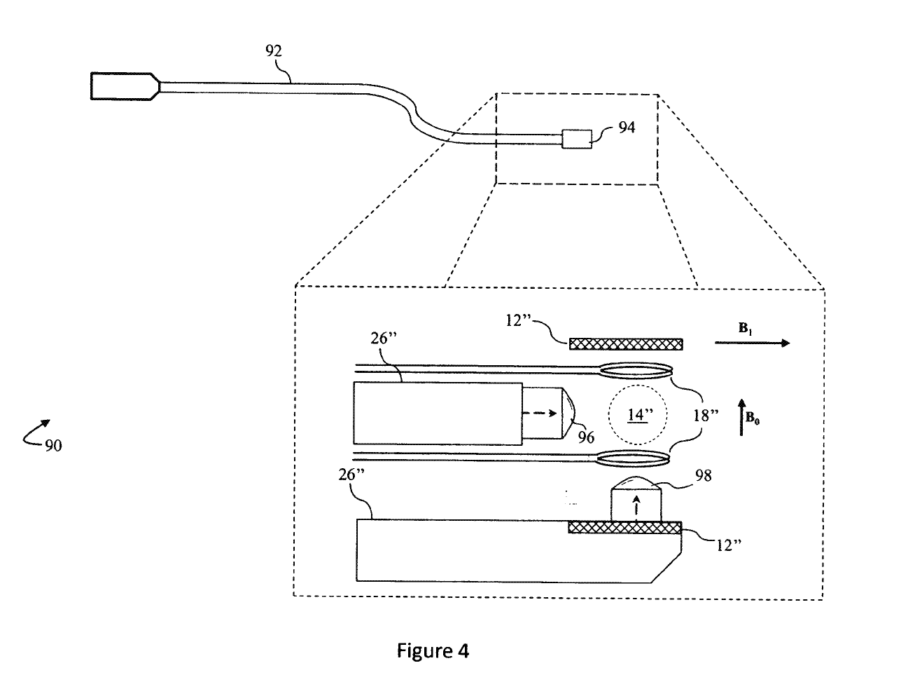

FIG. 4 is a diagrammatic illustration of another embodiment of a hyperpolarization device entirely contained within an invasive device;

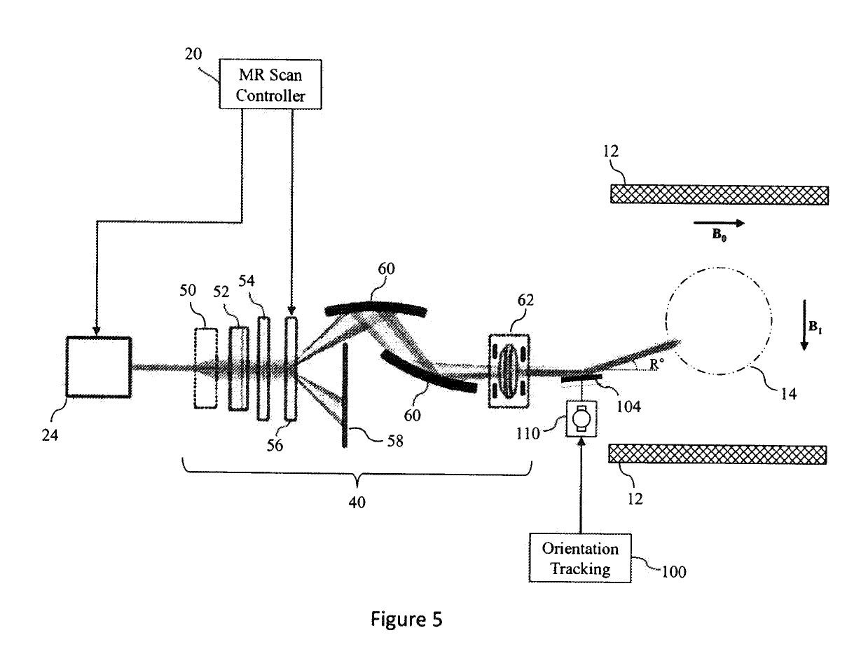

FIG. 5 is a diagrammatic illustration of another embodiment of a hyperpolarization device with an orientation modifier; and

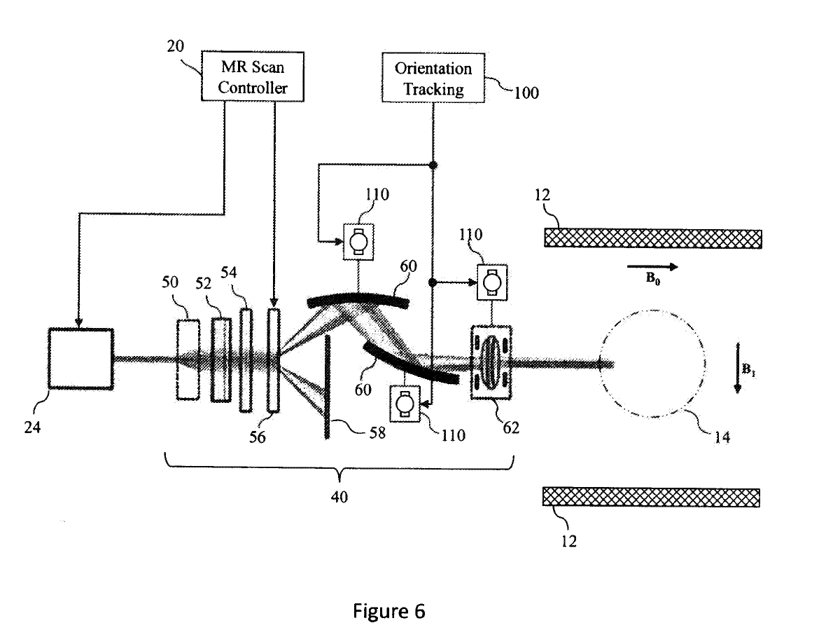

FIG. 6 is a diagrammatic illustration of another embodiment of a hyperpolarization device with selectively movable components.

Orbital angular momentum (OAM) is an intrinsic property of all azimuthal phase-bearing light, independent of the choice of axis about which the OAM is defined. When interacting with an electronically distinct and isolated system, such as a free atom or molecule, OAM can be transferred from the light to the center of mass of motion.

Various experiments used the interaction of OAM endowed light with matter, for example, optical tweezers, high throughput optical communications channels, optical encryption techniques, optical cooling, entanglements of photons with OAM, and entanglement of molecule quantum numbers with interacting photons' OAM. Because angular momentum is a conserved quantity, the OAM of absorbed photons is transferred in whole to interacting molecules. As a result, affected electron states reach saturation spin states, angular momentum of the molecule about its own center of mass is increased and oriented along the propagation axis of the incident light, and magnetons precession movement of the molecules are also oriented along the propagation axis of the incident light. These effects make it possible to hyperpolarized nuclei within fluids by illuminating them with light carrying spin and OAM.

An analysis of electromagnetic (EM) fields shows that there is a flow of EM energy with a first component that travels along the vector of the beam propagation, and a second component of EM energy that rotates about the axis of the beam propagation. The second component is proportional to the angular change of the potential vector around the beam propagation. This is signification because the rotational energy flow is proportional to the “l”, the OAM value, and the rotational energy transferred to the molecules with which the light interaction is increase with the value of the OAM.

When light carrying spin and OAM is absorbed by molecules, the angular momentum is conserved and the total angular momentum of the system (both the radiation and the matter) is not changed during absorption and emission of the radiation. When a photon is absorbed by an atom, the resulting angular momentum of the atom is equal to the vector sum of its initial angular momentum plus the angular momentum of the absorbed photon.

When a photon interacts with a molecule, only the OAM of the electrons is directly coupled to the optical transitions. The different types of angular momentum are coupled to each other by various interactions such as spin-orbit, spin rotation, hyperfine, OAM-rotation, and the like. The polarization of the photon flows through the electron orbital to molecule's the nuclear spin, electron spin, and molecular spin via these interactions. The magnitude of the interaction between the photon and the molecule is proportional to the OAM of the photon. Resultantly, the molecular moment aligns in the direction of the propagation axis of the incident light endowed with spin and OAM proportional to that of the OAM content of the incident light.

It is understood that any electromagnetic radiation can be endowed with

OAM, not necessarily only visible light. The described embodiment uses visible light, which interacts with the molecules of living tissue without any damaging effects; however, light/radiation above or below the visible spectrum, e.g. infrared, ultraviolet, x-ray, or the like, is also contemplated.

In one embodiment, the EM photonic beam endowed with OAM is used to replace the B₁ field of an existing scanner. In this embodiment, the photonic OAM beam is focused at a desired imaging or spectroscopy location such that the direction of the beam propagation is perpendicular to the B₀ field, so that the nuclei are hyperpolarized in an excited state. The light endowed with OAM is removed from the region on interest, and a standard MR signal is emitted as the nuclei relax into alignment with the B₀ field. In such an embodiment, resonance signals would only be received from dipoles accessible by the optical deliver system.

With reference to FIG. 1, in a first embodiment in which dipoles of interest are aligned with a magnetic field, a magnetic resonance imaging or spectroscopy system 10 includes a main magnet 12 which generates a temporally uniform B₀ field, e.g. 1.5 T, through an examination region 14. The main magnet can be an annular or bore-type magnet, a C-shaped open magnet, other designs of open magnets, or the like. Gradient magnetic field coils 16 disposed adjacent the main magnet serve to generate magnetic field gradients along selected axes relative to the B₀ magnetic field. A radio frequency receive coil, such as a whole-body radio frequency coil 18 is disposed adjacent the examination region. Optionally, local surface RF coils 18′ are provided in addition to or instead of the whole-body RF coil 18.

A scan controller 20 controls a gradient controller 22 which causes the gradient coils to apply selected magnetic field gradient pulses across the examination region, as may be appropriate to a selected magnetic resonance imaging or spectroscopy sequence. The scan controller 20 also controls an electromagnetic radiation source 24 which causes a photon based hyperpolarization device 26, which will be described in further detail, to emit an OAM endowed photon beam to directly hyperpolarize nuclear spins in an orientation perpendicular to the B₀ field, acting as a B₁ field. The OAM endowed photons are used to excite and manipulate magnetic resonance in the examination region. The scan controller also controls an RF receiver 28 which is connected to the whole-body or local RF coils to receive magnetic resonance signals emanating from the imaging region. The scanner controller synchronizes the hyperpolarization device 26, gradient controller 22, and readout RF receiver 28 based on a predefined scanning sequence.

The received data from the receiver 28 is temporarily stored in a data buffer 30 and processed by a magnetic resonance data processor 32. The magnetic resonance data processor can perform various functions as are known in the art, including image reconstruction, magnetic resonance spectroscopy, catheter or interventional instrument localization, and the like. Reconstructed magnetic resonance images, spectroscopy readouts, interventional instrument location information, and other processed MR data are displayed on a graphic user interface 34. The graphic user interface 34 also includes a user input device which a clinician can use for controlling the scan controller 20 to select scanning sequences and protocols, and the like.

In the illustrated embodiment, the hyperpolarization device 26, which includes an endowment arrangement 40 for endowing photons with OAM and an EM radiation source 24, is embodied as a catheter 44. However, other minimally invasive devices such as needles, endoscopes, laparoscopes, electronic pill, or the like are also contemplated. The EM radiation source is conveniently located outside of the catheter and fiber optics are used to channel the photons to the endowment arrangement 40. In another embodiment, the EM radiation source 24 is disposed adjacent to the endowment arrangement 40 adjacent a tip of the catheter. The catheter is inserted into the subject, e.g. via the femoral artery, and advanced to the region of interest. The application of the OAM endowed photons transverse, or substantially transverse, to the B₀ field causes the aligned dipoles to reach an excited state. Upon the removal of the OAM endowed photon beam, the excited dipoles precess back into alignment with the B₀ field and emit a magnetic resonance signal.

The emission of the OAM endowed photons can be controlled by that scan controller 20 in a number of ways. For example, the light source 42 is directly controlled by the scanner controller 20 or a mechanical shutter (not shown) at the distal end of the catheter can be controlled by the scanner controller 20 to selectively block the OAM endowed photon beam. The induced resonance signals are received by the external RF coils 18, 18′. It should be noted that an RF coil disposed at the distal end of the catheter is also contemplated.

The induced resonance signal can be spatially encoded in various ways. In one embodiment, the resonance is excited in and detected from a single voxel at a time. In another embodiment, the gradient magnetic field coils 16, arranged externally or disposed in proximity to the endowment arrangement 40, are configured to phase and frequency encode the resonance signal.

In another embodiment, the hyperpolarization device 26 is embodied as a transdermal surface probe that carries the endowment arrangement for endowing phtons with OAM. The surface probe can be pressed externally against a vein or artery, particularly one adjacent the skin, where it is sufficiently close that the OAM endowed photon beam will penetrate to the blood vessel. Other forms of EM radiation have a greater ability to penetrate through tissue enabling the vessels and arteries to be further from the surface. The nuclei of molecules in the blood flowing past the device are hyperpolarized and imaged as they flow through the subject's bloodstream. The images can illustrate penetration of blood into brain tissue, arterial tissue, venous flow, and the like.

With reference to FIG. 2, the endowment arrangement 40 is illustrated. In one embodiment, an endowment arrangement for endowing light with OAM, includes a white light source 24 that produces a visible white light that is sent to a beam expander 50. After the beam expanded, the light beam is circularly polarized. A linear polarizer 52 gives the unpolarized light a single linear polarization. A quarter wave plate 54 circularly polarizes the linearly polarized beam by shifting the phase of the linearly polarized light by ¼ wavelength. Using circularly polarizing light has the added benefit of polarizing electrons.

The circularly polarized light is passed through a phase hologram which imparts OAM and spin to an incident beam. The phase hologram maybe physically embodied in a spatial light modulator 56 as a liquid crystal on silicon (LCoS) panel, or it can be embodied in other optics, such as combinations of cylindrical lens or wave plates, or as a static phase hologram. The scanner controller 20 can control the LCoS panel to change the OAM value imparted onto the incident light during a scanning sequence. By tuning the spectral content of the light source and the amount of OAM imparted onto the incident EM radiation, the excitation can be configured to excite multiple species simultaneously such as molecular species like water, fat, or the like, multiple atomic species like Hydrogen, Carbon, Oxygen, Nitrogen, Phosphorous, or the like, and any combination thereof. The excitation can also be configured so that different molecular or atomic species are excited to different degrees or so that only the desired nuclei are excited. The OAM endowed EM radiation is adjusted and also used to manipulate magnetic resonance, e.g., induce spin or other echoes, dephase resonance, and the like.

A spatial filter 58 is placed after the phase hologram to selectively block 0^{th} order diffracted beams, and allows light with only one OAM value to pass. Since OAM of the system is conserved, it would be counterproductive to let the entire light pass, because the net OAM transferred to the target molecule would be zero. The diffracted beams with OAM are collected using concave mirrors 60 and focused on to the region of interest (ROI) with an objective lens 62. Alternatively, the mirrors may not be necessary if coherent light is employed. Furthermore, the lens may be replaced or supplemented with an alternate light guide, fiber optics, or the like.

With reference to FIG. 3, in another embodiment, the hyperpolarizing device 26′ is contained entirely within an invasive device 70 or a handheld surface probe. The illustrated embodiment depicts a catheter system; however, other invasive devices such as needles, laparoscopes, endoscopes, electronic pills, or the like are also contemplated. The catheter system includes an elongate portion 72 and a working end 74.

The working end 74 of the catheter system includes a hyperpolarization device 26′ for endowing a photon beam with OAM. The OAM endowed photons coming from the hyperpolarization device encounters a partially mirrored plate 76 that allows a portion of the photon beam to pass to a first objective lens 78. The first objective lens 78 is oriented orthogonal to a static magnetic field B₀, defined by magnets 12′ which act to polarize selected dipoles in an examination region 14″. Another portion of the photon beam is reflected to a first mirror 80 and onto a second mirror 82 where it then passes through a second objective lens 84, which is oriented orthogonally to the first objective lens 78 and parallel to the static magnetic field B₀. A mechanical shutter 86 acts to selectively block the orthogonally oriented EM radiation when it is not desired. Thus, the photon beam from the second objective lens acts to enhance the static magnetic field B₀ defined by magnets 12′, while the endowed photons from the first objective lens acts as a B₁ magnetic field to selectively optically excite the polarized dipoles into an excited state. When the orthogonally oriented photon beam is removed via the mechanical shutter 86, the excited dipoles precess back into alignment with the B₀ magnetic field and emit a resonance signal that is detected by RF receive coils 18′ which are operatively connected to the RF receiver 28.

With reference to FIG. 4, in another embodiment, a plurality of hyperpolarizing devices 26″ are contained entirely within an invasive device 90 or a handheld surface probe. The illustrated embodiment depicts a catheter system; however, other invasive devices such as needles, laparoscopes, endoscopes, electronic pills, or the like are also contemplated. The catheter system includes an elongate portion 92 and a working end 94.

The working end 94 of the catheter system includes two hyperpolarization device 26″ oriented orthogonal to one another for endowing a photon beam with OAM. The photons endowed with OAM, by one of the two hyperpolarization devices 26″, passes through a first objective lens 96 that is oriented orthogonal to a static magnetic field B₀, defined by magnets 12″ which act to polarize selected dipoles in an examination region 14″. The photons endowed with OAM, by the other hyperpolarization device 26″, passes through a second objective lens 98 that is oriented parallel to the static magnetic field B₀. Thus, the EM radiation from the second objective lens acts to enhance the static magnetic field B₀ defined by magnets 12″, while the EM radiation from the first objective lens acts as a B₁ magnetic field to selectively optically excite the polarized dipoles into an excited state and manipulates the excited resonance. When the orthogonally oriented photon beam is removed, the excited dipoles precess back into alignment with the B₀ magnetic field and emit a resonance signal that is detected by RF receive coils 18″ which are operatively connected to the RF receiver 28.

With reference to FIG. 1, to maintain an optimal orientation of the photon beam endowed with OAM relative to the B₀ field and/or B₁ field, the orientation of the endowed EM radiation is tracked by an orientation tracking unit 100. During an imaging procedure, as the hyperpolarization device is positioned in or adjacent to the region of interest, the endowment arrangement 40 may not be properly aligned to an optimal or desired orientation. To effectively enhance and/or replace either the B₀ or B₁ fields, the endowed photons is oriented parallel to the respective magnetic field. If not properly oriented, unexpected excited spins arises resulting in unwanted resonance which can affect image quality.

The orientation tracking unit 100 determines the spatial orientation of the endowed photon beam according to at least one orientation tracker 102 that is disposed on or in close proximity to the endowment arrangement 40. The orientation tracker 102 provides feedback to the orientation tracking unit 100 that is characteristic of the orientation of the endowment arrangement 40 relative to a predefined external coordinate system which coincides with the direction of the B₀ field. Prior to an imaging procedure, the hyperpolarization device 26, more specifically the orientation tracker 102, is registered or calibrated to the external coordinate system or to the optimal orientation prescribed by the imaging sequence. It should also be appreciated that frameless registration is also contemplated.

Once the relative orientation) (R°) of the hyperpolarization device 26 is known, it is tracked during the imagine procedure or intervention. During certain imaging procedures or interventions, the location of the region of interest can constrain the orientation of the hyperpolarization device 26 such that the optimal orientation cannot be achieved. An orientation modifier 104, disposed between the endowment arrangement 40 and the region of interest to be hyperpolarized, compensates for this misalignment by steering the endowed photon beam to the optimal orientation. The orientation tracking unit 100 determines a difference between the actual orientation and the optimal orientation, i.e. the relative orientation, and sends a signal to the orientation modifier 104 to steer the endowed photon such that it is parallel with either the B₀, B₁ field, or both as in the embodiment with two hyperpolarization devices 26″.

In one embodiment, the orientation tracker 102 provides an active signal to the orientation tracking unit 100 which is characteristic of the orientation of the endowment arrangement 40 relative to a predefined external coordinate system. For example, the active signal may be generated by one or more accelerometers, a gyroscope, a magnetometer, an RF tracking module, or any combination thereof.

In another embodiment, the orientation tracking unit 100 determines the orientation passively by monitoring a pattern of MRI visible fiducial markers in reconstructed image representations of the region of interest. By measuring dimensions of the pattern at various perspectives, e.g. along the axes of the predefined external coordinate system, the tracking unit 100 can determine the relative orientation and location of the endowment arrangement 40 and thus the relative orientation of the endowed photon beam. Alternatively, the orientation tracking unit 100 measures dimensions of the hyperpolarization device 26 and/or the endowment arrangement 40, in reconstructed image representations of the region of interest, at various perspectives to determine the relative orientation of the endowed photon beam.

In another embodiment in which the hyperpolarization device 26 delivers the endowed photon beam transdermally, the hyperpolarization device or at least the endowment arrangement 40 is supported by a pivotally segmented robotic arm which adjusts to position the hyperpolarization device adjacent to the region of interest. A joint connects two segments of the robotic arm and has multiple degrees-of-freedom (DOF). Each joint includes an encoder for each DOF which measures rotation or displacement. After the robotic arm is registered or calibrated to the predefined external coordinate system, the orientation tracking unit can determine the relative orientation of the endowed photon beam based on signals from each encoder of each joint.

After the relative orientation is determined, the orientation tracking unit 100 controls the orientation modifier 104 to steer or modify the orientation of the endowed photon beam emitted from the endowment arrangement 40 according to the determined relative orientation. The type of orientation modifier is the based on the wavelength of the electromagnetic radiation that is endowed and acts to redirect the photon beam while preserving the OAM.

With reference to FIG. 5, in one embodiment, the orientation modifier 104 includes an actuatable reflective surface, e.g. a mirror or the like, to steer an emitted beam of hyperpolarized EM radiation in or near the visible light spectrum, such as ultraviolet, infrared, or the like. Alternatively, when the EM radiation is in the X-ray range, an actuatable diffraction grating is used instead of the reflective surface. The actuation is provided by non-ferromagnetic actuators 110, such as a piezoelectric motor or the like. Alternatively, the actuation can be provided manually by a clinician by pushing or pulling wires that travel along the length of an interventional device.

In another embodiment, the orientation modifier 104 is a micro-mirror array which steers the endowed photon beam. The array includes a plurality of cantilevered micro-mirrors each actuated by a piezoelectric actuator. Alternatively, the micro-mirrors can be actuated by an electrostatic potential.

With reference to FIG. 6, in another embodiment, the emitted hyperpolarized beam is steered by actuating the concave mirrors 60 and the objective lens 62 (FIG. 2) of the endowment arrangement 40 in concert using non-ferrous actuators 110, such as a piezoelectric motor or the like.

In another embodiment, the non-ferrous actuator 110 re-orients the endowment arrangement 40 relative to the catheter.

Power to the actuator(s) can be provided by the orientation tracking system 100 via power wires that the travel along the length of the interventional device or a battery in close proximity to the actuator which eliminates a risk of inductive heating of the power wires. The battery may be charged inductively by the RF and/or gradient systems.

In another embodiment, the pivotally segmented robotic arm modifies the spatial orientation of at least the endowment arrangement 40 to modify the spatial orientation of the endowed photon beam. Each joint includes a non-ferromagnetic servo which can rotate or displace each segment selectively while each encoder monitors the position of a corresponding segment.

In another embodiment, parameters of the scanning sequence, such as the flip angle or the like, are adjusted to compensate for a relative orientation of the endowed photon beam that is not zero. The flip angle is the rotation of the net magnetization vector by the B₁ excitation pulse relative to the B₀ static magnetic field. In one MR scanning sequence, the flip angle is 90° to excite the polarized nuclei transverse to the B₁ field. In an example where the scanning sequence prescribes the OAM endowed photon beam to be parallel to the B₀ field, if the relative orientation of the photon beam is greater than zero then a net magnetization results in the transverse direction. To rotate the hyperpolarized nuclei orthogonally to the B₁ field, the flip angle is increased or decreased based on the relative orientation of the photon beam. For example, if the relative orientation angle is −6° then the adjusted flip angle is 96°; if the relative orientation angle is +8° then the resulting adjusted flip angle is 82°. The scanner controller 20 receives the relative orientation of the endowed photon beam and adjusts the flip angle of the prescribed imaging sequence accordingly. The advantage of this arrangement can reduce scan times and reduce inductive loading from the patient by reducing the duration of the B₁ magnetization field.

In another embodiment, the graphical user interface 34 displays an indicator characteristic of the relative orientation of the endowed photon beam. The clinician can adjust the orientation of the hyperpolarization device 26, 26′, 26″ manually by manipulating the device. In procedures where the region of interest is easily accessible and the hyperpolarization device has a wide range of motion in or near the region of interest, the clinician may elect to the manually manipulate the relative orientation. Alternatively, the hyperpolarization device may not include an orientation modifier 104 and feedback regarding to the relative orientation display visually for the clinician. This arrangement may reduce manufacturing costs and complexity.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A magnetic resonance system, comprising a main magnet which generates a static magnetic field B₀ in an examination region to polarize dipoles; and
a hyperpolarization device which directly hyperpolarizes nuclear spins via electromagnetic (EM) radiation endowed with orbital angular momentum (OAM), the hyperpolarization device hyperpolarizes nuclear spins relative to the static magnetic field B₀.

2. The magnetic resonance system according to claim 1, wherein
The hyperpolarized EM radiation is directed offset from, preferably orthogonal to, the B₀ field such that the EM radiation endowed with OAM acts to excite and/or manipulate magnetic resonance.

3. The magnetic resonance system according to claim 1, wherein the hyperpolarization device is configured to excite a plurality of distinct nuclear species simultaneously.

4. The magnetic resonance system according to claim 1, further comprising:
an RF system which generates a transverse magnetic field B₁ to induce and manipulate magnetic resonance signals in the examination region; and/or receives the induced magnetic resonance signals from the examination region.

5. The magnetic resonance system according to claim 1, wherein the examination region is in vivo and further comprising:
an interventional device which is insertable into a patient, the interventional device is configured to position the hyperpolarization device adjacent to the examination region in vivo.

6. The magnetic resonance system according to claim 1, further comprising:
a transdermal surface probe which outputs light endowed with OAM from the hyperpolarization device to penetrate tissue of a patient.

7. The magnetic resonance system according to claim 4, further comprising:
a gradient magnetic field system which spatially encodes the induced magnetic resonance signals; and
a scanner controller which synchronizes the hyperpolarization device, RF system, and gradient magnetic field system to perform a predefined scanning sequence.

8. The magnetic resonance system according to claim 1, further including:
a second hyperpolarization device which directly hyperpolarizes nuclear spins via OAM offset from, preferably orthogonal to, the first hyperpolarization device.

9. The magnetic resonance system according to claim 1, further including:
an orientation tracking system which determines a spatial orientation of the EM radiation endowed with OAM relative to a predefined external coordinate system.

10. The magnetic resonance system according to claim 9, wherein the orientation tracking system includes:
at least one orientation modifier which adjusts the spatial orientation of the EM radiation endowed with OAM without altering endowed OAM according to a desired orientation relative to the predefined external coordinate system.

11. The magnetic resonance system according to claim 10, wherein the orientation modifier includes at least one of:
a selectively movable mirror;
a selectively movable diffraction grating;
a selectively movable objective lens;
a controllable micro-mirror array; and
a pivotally segmented robotic arm.

12. The magnetic resonance system according to claim 9, wherein orientation tracking unit is configured to control the orientation modifier to direct the EM radiation endowed with OAM to the desired orientation based on a detected orientation of the hyperpolarization device relative to the predefined external coordinate system.

13. The magnetic resonance system according to claim 9, wherein the scanner controller is configured to control at least one parameter of a predefined scanning sequence based on the detected orientation of the hyperpolarization device.

14. The magnetic resonance system according to claim 1, wherein the hyperpolarization device includes:
an EM radiation source which provides the EM radiation, such as visible light, ultra-violet, infra-red, x-ray, or the like, to be endowed with OAM; and
an endowment arrangement which endows the EM radiation with OAM and directs the endowed light to a region of interest to be hyperpolarized.

15. A method for magnetic resonance, including generating a static magnetic field (B₀) through an examination region to polarize dipoles; and
directly hyperpolarizing nuclear spins via electromagnetic (EM) radiation endowed with orbital angular momentum (OAM) relative to the static magnetic field B₀.

16. The method according to claim 15, wherein the EM radiation endowed with OAM is introduced to a region of interest angularly offset from, preferably orthogonal to, the static magnetic field to induce and/or manipulate magnetic resonance.

17. The method according to claim 15, further including:
controlling a spectral content of the EM radiation and/or an amount of OAM imparted onto the EM radiation, e.g. to excite resonance in a plurality of distinct species simultaneously.

18. The method according to claim 15, further including:
tracking the spatial orientation of the EM radiation endowed with OAM relative to a predefined external coordinate system.

19. The method according to claim 18, further including at least one of:
modifying the spatial orientation of the EM radiation endowed with OAM without altering the OAM according to a desired orientation relative to the predefined external coordinate system; and
adjusts based on the detected orientation of the hyperpolarization device at least one of a magnetic resonance sequence which induce magnetic resonance signals and processing of the induced magnetic resonance signals.

20. A hyperpolarization device, comprising:
an electromagnetic (EM) radiation source which provides the EM radiation, such as light or x-rays, to be endowed with OAM;
an endowment arrangement which endows the EM radiation with OAM and directs the OAM endowed EM radiation to a region of interest to be hyperpolarized;
an orientation tracking system which determines a spatial orientation of the directed endowed light relative to a predefined external coordinate system; and at least one of.
a mechanical arrangement which adjusts a direction in which the OAM endowed EM radiation is directed,
a processor which adjusts at least one of a magnetic resonance sequence and processing of resonance data generated by the resonance sequence.
